SEQUENCE LISTING (1 sequence)

SEQ ID NO: 1 - protein, 29 aa, Artificial Sequence
  Description of Artificial Sequence Synthetic peptide
YADAIFTNSYRKVLGQLSARKLLQDIMSR

(19) United States
(12) Patent Application Publication
(10) Pub. No.: US 2024/0181015 A1
(43) Pub. Date: Jun. 6, 2024

(54) EFFECT OF GHRH ANTAGONISTS IN DIABETES AND OBESITY

(71) Applicants: AUGUSTA UNIVERSITY RESEARCH INSTITUTE, INC., Augusta, GA (US); UNIVERSITY OF MIAMI, Miami, FL (US); THE UNITED STATES GOVERNMENT AS REPRESENTED BY THE DEPT. OF VETERANS AFFAIRS, Washington, DC (US)

(72) Inventors: Neal L. WEINTRAUB, Augusta, GA (US); Maritza J. ROMERO LUCAS, Augusta, GA (US); Rudolf LUCAS, Augusta, GA (US); Andrew SCHALLY, Miami, FL (US); Renzhi CAI, Miami, FL (US)

(73) Assignees: AUGUSTA UNIVERSITY RESEARCH INSTITUTE, INC., Augusta, GA (US); UNIVERSITY OF MIAMI, Miami, FL (US); THE UNITED STATES GOVERNMENT AS REPRESENTED BY THE DEPT. OF VETERANS AFFAIRS, Washington, DC (US)

(21) Appl. No.: 18/554,144

(22) PCT Filed: Apr. 4, 2022
(86) PCT No.: PCT/US2022/023326

§ 371 Date: Oct. 5, 2023

Related U.S. Application Data
(60) Provisional application No. 63/170,919, filed on Apr. 5, 2021.

(51) Int. Cl. A61K 38/25; A61P 3/04; A61P 3/10

(52) U.S. Cl. CPC A61K 38/25; A61P 3/04; A61P 3/10

(57) ABSTRACT

Provided herein are compositions and methods of use thereof for the treatment of metabolic disease such as diabetes and obesity. One embodiment provides a method of treating a metabolic disease or syndrome in a subject in need thereof by administering to the subject a therapeutically effective amount of a growth hormone releasing hormone (GHRH) antagonist or a pharmaceutical composition comprising the GHRH antagonist to reduce triglyceride-rich-lipoproteins (TRL) in the subject to treat the metabolic condition or syndrome.

### CROSS-REFERENCE TO RELATED APPLICATIONS

This invention claims the benefit of priority to U.S. Provisional Application No. 63/170,919, filed on Apr. 5, 2021, the contents of which are incorporated herein by reference.

### SEQUENCE LISTING

The instant application contains a Sequence Listing which has been submitted electronically in ASCII format and is hereby incorporated by reference in its entirety. Said ASCII copy, created on Mar. 31, 2022, is named 064466_141PCT_SL.txt and is 1 kilobyte in size.

### FIELD OF THE INVENTION

Aspects of the invention are generally directed compositions and method for treating metabolic disorders including diabetes and obesity.

### BACKGROUND OF THE INVENTION

Diabetes is the fifth-leading cause of death in the US, with mortality rates increasing by 45% since 1987. From 1980 to 2010, the prevalence of diabetes has increased by 300%, according to data from the Center for Disease Control and Prevention (Roger et al., Circulation 2011, 128, e18-e209). Further, according to the National Diabetes Statistics Report (2014), an estimated 29.1 million Americans are afflicted with diabetes mellitus, 8.1 million of whom are undiagnosed. Type 1 diabetes, which used to be called juvenile onset diabetes, occurs in 3-5% of diabetics. In addition, it is estimated that about 10% of type 2 insulin-requiring diabetics may actually be adult-onset type 1. Type 2 diabetes (TD2M) comprises 80-95% of all diabetics. This type of diabetes used to be called adult or maturity onset diabetes. Secondary diabetes, in which the diabetes is a secondary manifestation of another ailment, comprises the rest.

Currently, the only treatment option for patients with type 1 diabetes is insulin injections. The first line intervention for patients with type 2 diabetes is focused on dietary modification, lifestyle changes, and oral hypoglycaemic agents. However, if these strategies fail to regulate blood glucose, patients become reliant on exogenous insulin to maintain their blood glucose levels. Many studies have shown that exercise improves glycemic control in patients with type 2 diabetes (Baum et al., Int. J. Med. Sci. 2007, 4, 159-163; Davis et al., S. D. Med. 2012, 65, 35-37). This beneficial effect is likely multifactorial, including increasing energy expenditure as well as insulin-induced membrane translocation of GLUT4. In addition, a number of studies have shown that exercise improves glycemic control in T2DM patients and in animal models, in part, due to anti-inflammatory properties (Teixeira-Lemos et al., Cardiovasc. Diabetol. 2011, 28, 12; de Lemos et al., Med. Sci. Monit. 2007, 13, BR168-BR174; Teixeira de Lemos et al., Nutrition 2009, 25, 330-339). However, sustained exercise routines are strenuous, time consuming, and difficult to maintain given today's rapid paced digital society (Unick et al., Med. Sci. Sports Exerc. 2010, 42, 745-753). There is a great need to increase the efficiency and compliance of conventional exercises, achieving the euglycemic and other health benefits of exercise without the vigor and duration of standard exercise intervention programs (Brownell, Annu. Rev. Public Health 1986, 7, 521-533).

Cardiovascular disease (CVD) is the leading cause of morbidity and mortality in type 1 diabetes mellitus (T1D) and type 2 diabetes mellitus (T2D) (Lee et al., Ther. Adv. Chronic Dis. 2015, 6, 347-374; Moreno et al., Circulation 2000, 102, 2180-2184). It is estimated that the risk of CVD in diabetes mellitus (DM) is 2 to 4 times higher than in the general population (Raghavan et al., J Am. Heart. Assoc. 2019, 8, e011295; Alabas et al., J. Epidemiol. Community Health 2016, 207402). Much of this increased risk is thought to be related to the development of an atherogenic lipid profile, in which hypertriglyceridemia is an essential component (Toth et al., Vasc. Health Risk Manag. 2016, 12, 171-183).

Triglyceride-rich-lipoproteins (TRL) generated in the small intestine as chylomicrons (CM) and in the liver as very low-density lipoprotein (VLDL) and their remnants represent an important risk factor for cardiovascular disease (CVD) in subjects with diabetes, obesity or the metabolic syndrome, independent of low-density lipoprotein (LDL) levels (Vallejo-Vaz et al., Circulation 2018, 117, 032318; Annuzzi et al., Nutr. Metab. Cardiovasc. Dis. 2008, 18, 531-538; Davidson et al., Eur. Heart J. 2018, 39, 620-622).

A critical barrier to progress in the treatment of obesity/diabetes-associated dyslipidemia is the identification of new therapeutic approaches to reduce TRL. Despite the efficacy and safety of LDL lowering therapies, this residual CVD risk remains a challenge that needs to be addressed by scientists and clinicians.

Recent studies highlight the role of the intestine in whole body lipid homeostasis (Xiao et al., Am. J Physiol. Endocrinol. Metab. 2011, 301, E429-446). Published data demonstrated a novel role for peripheral Growth Hormone Releasing Hormone Receptors (pGHRH-R) in a rodent model of type 1 diabetes (T1D)-associated dyslipidemia (Romeo et al., Proc. Natl. Acad. Sci. USA 2016, 113, 1895-1900). Expression of pGHRH-R is increased in the small intestine of rats injected with streptozotocin (STZ) to induce T1D. In addition, specific activation of pGHRH-R with the GHRH agonist JI-34, promotes secretion of apolipoprotein B48 (ApoB-48), a component of chylomicrons, in rat small intestine epithelial cells (IEC) loaded in vitro with oleic acid, a fatty acid that is naturally present in various animal and vegetable fats and oils. This effect is associated with dysfunction of the receptor for the incretin Glucagon-like peptide-1 (GLP-1R), which is expressed in the intestine and reduces TRL levels in vivo (Hein et al., Diabetes 2013, 62, 373-381; Sivertsen et al.; Nat. Rev. Cardiol. 2012, 9, 209-222). Administration of the GHRH antagonist MIA-602 reduced ApoB-48 lipoproteins both in STZ-rats and in rat IEC in vitro (U.S. patent Ser. No. 10/201,588).

Therefore, there remains a need for improved methods for the treatment of diabetes and obesity.

It is an object of the invention to provide Growth Hormone Releasing Hormone (GHRH) antagonists and methods of use thereof.

It is still another object of the invention to provide methods for treating diabetes and obesity.

### SUMMARY OF THE INVENTION

Compositions and methods of their use for treating metabolic disorders are provided. One embodiment provides compositions and methods for the treatment of diabetes, obesity, or both for example by reducing levels of triglyceride-rich-lipoproteins (TRL), for example serum levels of TRL, or reducing TRL bioactivity in the subject. TRL levels represent an important risk factor for cardiovascular disease (CVD) in subjects with diabetes, obesity or the metabolic syndrome, independent of low-density lipoprotein (LDL) levels (Marston et al., Circulation 2019, 140, 1308-1317; Ference et al., JAMA 2019, 321, 364-373; Bhatt et al., N. Engl. J. Med. 2019, 380, 11-22).

One embodiment provides treating a metabolic disorder or metabolic syndrome in subject in need thereof by administering to the subject a therapeutically effective amount of a growth hormone releasing hormone (GHRH) antagonist to treat the metabolic disorder. In some embodiments, the metabolic disorder or syndrome is treated by administering to a subject in need thereof a GHRH antagonist or a pharmaceutical composition comprising an effective amount of a GHRH antagonist, wherein the GHRH antagonist includes but not limited to MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof. The metabolic disorder or syndrome can be pre-diabetes, type I diabetes, type II diabetes, gestational diabetes, obesity, or a combination thereof.

Another embodiment provides a method of treating diabetes in a subject in need thereof, by administering to the subject a therapeutically effective amount of a GHRH antagonist or a pharmaceutical composition comprising a GHRH antagonist. In some embodiments, the diabetes is a pre-diabetic condition, type I diabetes, type II diabetes or gestational diabetes. In some embodiments, the growth hormone releasing hormone (GHRH) antagonist is selected from the group consisting of MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof.

In some embodiments, the therapeutically effective amount of the GHRH antagonist or pharmaceutical composition comprising the GHRH antagonist is administered parenterally or enterally including, but not limited to, intravenously, intramuscularly, subcutaneously or orally. In some embodiments, the therapeutically effective amount of the GHRH antagonist is administered continuously or intermittently.

Another embodiment provides a method of treating obesity in a subject in need thereof by administering to the subject a therapeutically effective amount of a GHRH antagonist or pharmaceutical composition comprising the GHRH antagonist. In some embodiments, the GHRH antagonist is selected from the group consisting of MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof. In some embodiments, the therapeutically effective amount of the GHRH antagonist or pharmaceutical composition comprising the GHRH antagonist is administered parenterally or enterally including, but not limited to, intravenously, intramuscularly, subcutaneously or orally.

Another embodiment provides a method of reducing or inhibiting TRL activity in a subject in need thereof by administering an effective amount of a GHRH antagonist or a pharmaceutical composition comprising the GHRH antagonist to reduce or inhibit TRL activity in the subject. In some embodiments, the GHRH antagonist is selected from the group consisting of MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof. In some embodiments, the therapeutically effective amount of the GHRH antagonist or pharmaceutical composition comprising the GHRH antagonist is administered parenterally or enterally including, but not limited to, intravenously, intramuscularly, subcutaneously or orally. The GHRH antagonist can be administered parenterally or enterally including, but not limited to, intravenously, intramuscularly, subcutaneously or orally.

### BRIEF DESCRIPTION OF THE DRAWINGS

FIGS. 1A and 1B show peripheral growth hormone releasing hormone receptor (pGHRH-R) and GHRH tissue levels in normal wild type C57BL/6 male mice. FIG. 1A is a Western blot of mouse duodenum, jejunum-ileum, and liver extracts showing expression of pGRH-R. FIG. 1B is a bar graph showing growth hormone releasing hormone (GHRH) (pg/mg protein) and the ligand of pGHRH-R from mouse duodenum, jejunum-ileum, and liver extracts measured by ELISA.

FIGS. 2A, 2B and 2C show the effect of the treatment with MIA-602 on triglycerides, ApoB-48, and very low density lipoprotein (VLDL) levels, respectively, in db/db mice. FIG. 2A shows a significant increase of total triglycerides (mg/dl) in untreated db/db mice versus MIA-602 treated db/db mice. FIG. 2B shows a significant increase of total ApoB-48 (ng/ml) in untreated db/db mice versus MIA-602 treated db/db mice. FIG. 2C shows a significant increase of total VLDL (mg/dl) in untreated db/db mice versus MIA-602 treated db/db mice. Treatment with MIA-602 significantly reduced all lipid abnormalities in db/db mice as compared untreated db/db mice.

FIGS. 3A, 3B and 3C show microsomal triglyceride transfer protein (MTTP) activity and ApoB-48 levels in protein extracts from the jejunum of db/db mice treated with the GHRH antagonist MIA-602. FIG. 3A is a bar graph showing no significant changes in the microsomal triglyceride transfer protein (MTTP) activity (pmole/μg protein/hour) among the groups, indicating the lowering effect of GHRH antagonist MIA-602 on plasma lipid levels observed in db/db mice, was not associated with significant changes in the activity of microsomal triglyceride transfer protein (MTTP) at least in the small intestine. FIG. 3B is a representative blot of ApoB-48 expression in protein extracts from jejunum of lean mice, db/db mice, db/db mice treated with GHRH antagonist MIA-602 and lean mice treated with GHRH antagonist MIA-602. FIG. 3C is a bar graph showing densitometric analysis (optical density O.D.) of ApoB-48 corrected by protein loading (vinculin), and demonstrates a significant increase of ApoB-48 content in jejunum segments of small intestine from db/db mice as compared to lean control groups. It also shows that GHRH antagonist MIA-602 (25 μg/kg/day, s.c.) significantly reduced ApoB-48 content in small intestine of db/db mice as compared to untreated db/db mice.

FIGS. 4A, 4B and 4C show the effect of MIA-602 on fasting blood glucose, Hemoglobin A1c (HbA1c), and glucagon levels in db/db mice. FIG. 4A is a bar graph showing significant increase of glucose level (mg/dl) in all db/db mice groups versus lean control. However, a modest but significant reduction in glucose level was seen in db/db mice treated with MIA-602 as compared to untreated db/db mice. FIG. 4B is a bar graph showing significant increase of hemoglobin A1c (HbA1c) (%) level in all db/db mice groups versus lean control. However, a modest but significant reduction in hemoglobin A1c (HbA1c) level was seen in db/db mice treated with MIA-602 as compared to untreated db/db mice. FIG. 4C shows a significant reduction in glucagon levels (pg/ml) in db/db mice treated with MIA-602 as compared to untreated db/db mice.

FIG. 5 is a bar graph showing diet-induced obesity (DIO) in mouse model. It exhibits that mice fed with high fat diet (HFD) for 12 weeks versus mice on control diet (CD). Mice fed on HFD demonstrated significant increase in body weight, and enhanced as well as fluctuating blood glucose levels in the fasting state as compared to mice fed on CD.

FIG. 6 shows the full length GHRHR gene map that includes nucleotide sequence from exon 1 to exon 13. The splice variants (SV) of GHRHR gene lack the first 3 exons. The bioactive SV-1, which has ligand-dependent but also independent activity, has the highest homology to the full receptor. SV1 has an intronic sequence (intron 3) corresponding to the translation initiation site followed by a nucleotide sequence derived from exon 4-13. The GHRHR floxed mice using embryonic ES Cells was generated. The ES cell clones were targeted with a floxed allele of GHRHR with exons 2-5 flanked by loxP sites, and were injected into C57BL/6-Albino blastocysts for chimera production and generation of GHRHR flox/wt mice. The colony was expanded and obtained the homozygous GHRHR flox/flox mice, which were cross bred with mice expressing Cre recombinase in the small intestine. Recombinase activity results in the deletion of loxP flanked targets in the intestine.

FIGS. 7A, 7B, 7C, 7D, 7E, 7F, 7G and 7H show micrographs of paraffin sections of small intestine processed for immunostaining of the receptor for GHRH expression of wild type (WT) mice fed on control diet (CD) at 20× magnification (FIG. 7A) and at 63× magnification (FIG. 7B), WT mice fed on high fat diet (HFD) at 20× magnification (FIG. 7C) and at 63× magnification (FIG. 7D), WT mice fed on high fat diet (HFD) with a genetic ablation of the receptor of GHRH in the epithelium of small intestine (GHRHR^{Fl/fl}/Vil-Cre⁺) at 20× magnification (FIG. 7E) and at 63× magnification (FIG. 7F), and WT mice fed on high fat diet (HFD) with negative control (no primary Ab) at 20× magnification (FIG. 7G) and at 63× magnification (FIG. 7H), respectively. The expression of the GHRH receptor was increased in the small intestine epithelial cells of the wild type mice fed on high fat diet (HFD), as compared to the mice fed on control diet (CD). This effect was not observed in mice with a genetic ablation of the receptor of GHRH in the epithelium of small intestine.

FIG. 8 is a graph showing postprandial triglyceride plasma levels demonstrating the effect of GHRH antagonist AVR-352 (Antago) or deletion of GHRH receptor in small intestine epithelium (HOMO) of mice fed on high fat diet (HFD) or control diet (Ctrl diet) for 16 weeks. AVR-352 (25 μg/kg/day, subcutaneous, every other day), was given to wild type mice after 12 weeks of high fat diet (HFD) for 4 weeks. Wild type mice fed a high fat diet had increased levels of triglycerides in the postprandial state versus fasting up to 2 hours after oral lipid loading, when compared with mice under control diet. This effect was significantly blunted by treatment with the GHRH antagonist AVR-352. The contribution of the GHRH receptor on increased triglyceride output from the small intestine, was unequivocally validated in mice with selective deletion of ghrhr gene in the epithelium of the small intestine. When these mice were fed on HFD or CD, the triglyceride levels after oral lipid loading were significantly reduced when compared to wild type mice fed on the same diets.

FIG. 9 shows effect of GHRH antagonist AVR-352 on fasting triglyceride levels in diet-induced obesity (DIO) in C57BL/6 mice. Total triglycerides measured in plasma showed significant higher levels in mice fed on high fat diet (HFD) as compared to mice fed on a normal control diet (CD) after 24 weeks. GHRH antagonist, AVR-352 significantly reduced triglycerides in HFD mice after 12 weeks of treatment when compared to untreated HFD mice.

FIGS. 10A, 10B, 10C and 10D represent Comprehensive Laboratory Animal Monitoring System (CLAMS) showing a) food intake, b) body weight gain, c) activity-ambient, and d) respiratory exchange ratio. FIG. 10A is a bar graph showing daily food intake (g/day) in MIA-602 treated HFD mice and HFD mice. No significant differences were observed among the groups. FIG. 10B is a bar graph showing body weight gain (g) in MIA-602 treated HFD mice and HFD mice. MIA-602 treated HFD mice had significantly lower body weight gain than untreated HFD mice. FIG. 10C is a bar graph showing activity-ambient (counts/day) in MIA-602 treated HFD mice and HFD mice. MIA-602 treated HFD mice had significantly higher daily activity-ambient than untreated HFD mice. FIG. 10D is a bar graph showing respiratory exchange ratio (RER) in MIA-602 treated HFD mice and HFD mice. MIA-602 treated HFD mice had significantly lower respiratory exchange ratio (RER) than untreated HFD mice.

FIGS. 11A, 11B and 11C show the effect of GHRH antagonist AVR-352 or ablation of GHRH receptor gene (HOMO) on ApoB-48 and MTTP content in small intestine of mice. Protein extracts were isolated from scrapped epithelium of whole small intestine of mice. FIG. 11A is a representative blot of ApoB-48 and MTTP protein expression, measured in protein extracts of scrapped epithelium of small intestine. FIG. 11B is a bar graph showing significant increase of ApoB-48 protein content in small intestine epithelium from wild type mice fed on HFD as compared to mice fed on control (Ctrl) diet. Mice treated with AVR-352 significantly reduced ApoB-48 content in small intestine of mice fed on HFD as compared to untreated mice fed on HFD. FIG. 11C is a bar graph showing significant increase of MTTP protein content in small intestine epithelium from wild type mice fed on HFD as compared to mice fed on control (Ctrl) diet. Mice treated with AVR-352 showed no effects on MTTP protein content in small intestine of mice fed on HFD as compared to untreated mice fed on HFD.

FIGS. 12A, 12B and 12C show receptor of Growth hormone releasing hormone (GHRH-R) was expressed in human small intestine epithelial cells (hulECs). FIG. 12A is a representative blot of expression of the receptor for GHRH measured in protein extracts isolated from hulECs grown in normal medium. FIG. 12B is a representative blot of GHRH-R expression in protein extracts of hulEC transfected for 96 hours with GHRH-R siRNA (10 nM, Dharmacon). As control siRNA, non-targeted scrambled (Sc) sequence was used for transfection (10 nM, Dharmacon). FIG. 12C is a bar graph illustrating densitometric analysis (optical density O.D., fold over Ctrl Sc siRNAS) of expression of the receptor of GHRH showed a significant decrease after transfection with GHRH-R siRNA as compared to cells transfected with no-targeting Sc siRNA. Values were corrected by protein loading (0-actin).

FIG. 13 is a schematic representation of the effect of the receptor of GHRH on ApoB-48 secretion in cultured human small intestine epithelial cells (hulEC).

FIG. 14 is a bar graph showing confluent monolayers of human small intestine epithelial cells (hulECs, ATCC) were cultured in 6-well plates and loaded or not with oleic acid after treatments. ApoB-48 was measured in culture supernatants. GHRH agonist JI-34 (1 μM, 1 h) impaired the actions of the GLP-1 receptor agonist exendin 4 (Ex-4, 10 nM, 2 h) on reducing the ApoB48 secretion upon loading hulEC with oleic acid (0.5 mM, 3 h). This effect was completely blunted by co-treatment of cells with the GHRH antagonist MIA-602 (1 μM, 1 h).

FIG. 15 is a bar graph showing confluent monolayers of human small intestine epithelial cells (hulECs, ATCC) were cultured in 6-well plates and loaded or not with oleic acid after treatments. ApoB-48 was measured in culture supernatants. GHRH agonist JI-34 (1 μM, 1 h) significantly increased the ApoB-48 secretion upon loading hulEC with oleic acid (0.5 μM, 3 h), as compared to non-treated cells (medium alone), or cells treated with the GLP-1 receptor agonist exendin 4 (Ex-4, 10 nM, 2 h). Similarly, native GHRH ligand significantly increased ApoB-48 secretion in a dose-dependent manner.

FIG. 16 is a bar graph showing confluent monolayers of human small intestine epithelial cells (hulECs, ATCC) were cultured in 24-well plates and transfected for 96 hs with GHRH-R siRNA (10 nM, Dharmacon). As control siRNA, non-targeting scrambled (Sc) sequence was used for transfection (10 nM, Dharmacon). Cells were loaded or not with oleic acid after treatments, and ApoB-48 was measured in culture supernatants. When cells had intact GHRH receptor after transfection with non-targeting Sc siRNA, the GHRH agonist JI-34 (1 μM, 1 h) significantly increased the ApoB-48 secretion upon loading hulEC with oleic acid (0.5 μM, 3 h). Under these conditions, JI-34 also impaired the actions of the GLP-1 receptor agonist exendin 4 (Ex-4, 10 nM, 2 h) on reducing the ApoB-48 secretion. These effects were completely blunted when hulEC were depleted of GHRH receptor after transfection with GHRH-R siRNA.

FIGS. 17A and 17B represent Comprehensive Laboratory Animal Monitoring System (CLAMS) showing a) body weight gain, and b) activity-ambient. FIG. 17A is a bar graph showing body weight gain (%) in AVR-352 treated HFD mice and HFD mice. AVR-352 treated HFD mice had significantly lower body weight gain than untreated HFD mice. FIG. 17B is a bar graph showing activity-ambient (counts/day) in AVR-352 treated HFD mice and HFD mice in activity-dark cycle and activity-light cycle. AVR-352 treated HFD mice had significantly higher daily activity-ambient than untreated HFD mice during the dark cycle. No significant differences were seen during the light cycle.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “A1C”, “HbA1C”, and “hemoglobin test” refer to a person's average levels of blood glucose over the past 3 months. The A1C test is the primary test used for diabetes management and diagnosis. The A1C test results are reported as a percentage. Normal A1C levels fall below 5.7%.

As used herein, “Type-2 diabetes” (T2DM) is a metabolic disease characterized by excessive hepatic glucose release, central obesity, impaired pancreatic insulin secretion and decreased insulin sensitivity by target cells leading to insulin resistance with chronic and persistent hyperglycemia. A subject with type-2 diabetes has an A1C higher than 6.5%.

As used herein, “prediabetes” refers to the condition in which a person's blood sugar level is higher than normal, but not high enough to be classified as type-2 diabetes. A person with prediabetes has an A1C between 5.7% and 6.4%. Prediabetes can progress into type-2 diabetes if the subject does not make lifestyle changes to control their blood glucose.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject can be a mammal. In some aspects, a subject can a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for pulmonary fibrosis, such as, for example, prior to the administering step. In some aspects of the disclosed methods, the “patient” has been diagnosed with a need for treatment for cancer, such as, for example, prior to the administering step.

As used herein, the term “amelioration” refers to a lessening of at least one indicator, sign, or symptom of an associated disease, disorder, or condition. The severity of indicators may be determined by subjective or objective measures, which are known to those skilled in the art.

The terms “Inhibit,” “inhibiting” and “inhibition” refer to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

The terms “treat,” “treating,” or “treatment” refer to alleviating, reducing, or inhibiting one or more symptoms or physiological aspects of a disease, disorder, syndrome, or condition. “Treatment” as used herein covers any treatment of a disease in a subject, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

### II. Embodiments

Compositions and methods for inhibiting or reducing TRL activity in a subject in need thereof are provided. One embodiment provides a method for treating diabetes, obesity, or both in a subject in need thereof by administering an effective amount of a GHRH antagonist to reduce or inhibit TRL activity in the subject. In some embodiments, the growth hormone releasing hormone (GHRH) antagonist are selected from the group consisting of MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof. In some embodiments, the therapeutically effective amount of the GHRH antagonist is administered parenterally or enterally including, but not limited to, intravenously, intramuscularly. In one embodiment, the diabetes is a pre-diabetic condition. In another embodiment, the diabetes to be treated is type I diabetes or type II diabetes. In yet another embodiment, the diabetes to be treated is a gestational diabetes. In another embodiment, the therapeutically effective amount of the GHRH antagonist is administered before and/or after mealtime. In another embodiment, the therapeutically effective amount of the GHRH antagonist is administered continuously. In one embodiment, the subject is a mammal. In other embodiment, the subject is a human.

In some embodiments the subject is either obese or at risk of obesity. In one embodiment, the subject has a body mass index (BMI) of over 25. In another embodiment, the subject has a body mass index (BMI) of between 25 and 30. In another embodiment, the subject has a body mass index (BMI) of over 30.

Another embodiment provides a method of reducing or inhibiting TRL activity in a subject in need thereof by administering an effective amount of a GHRH antagonist to reduce or inhibit TRL activity in the subject. In some embodiments, the growth hormone releasing hormone (GHRH) antagonist are selected from the group consisting of MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof. In some embodiments, the therapeutically effective amount of the GHRH antagonist is administered parenterally or enterally including, but not limited to, intravenously, intramuscularly, subcutaneously or orally. The GHRH antagonist can be administered parenterally or enterally including, but not limited to, intravenously, intramuscularly, subcutaneously or orally.

The amino acid sequences for exemplary GHRH antagonist MIA-602 is Tyr-Ala-Asp-Ala-Ile⁵-Phe-Thr-Asn-Ser-Tyr¹⁰-Arg-Lys-Val-Leu-Gly¹⁵-Gln-Leu-Ser-Ala-Arg²⁰-Lys-Leu-Leu-Gln-Asp²⁵-Ile-Met-Ser-Arg²⁹-NH2 (SEQ ID NO: 1).

MIA-602 can also be referred to as hGH-RH(1-29)NH2 analog. hGH-RH(1-29)NH2 I is considered a standard GHRH-R antagonist [Ac-Tyr¹,D-Arg²]. hGH-RH(1-29)NH2 is fragment of the native GH-RH. Synthetic analogs of GHRH based on the structure of hGH-RH(1-29)NH2 can be used in the methods disclosed herein. Examples of GHRH analogs are disclosed in U.S. Pat. No. 9,260,504 and are incorporated herein by reference. Also disclosed are variants of MIA-602 and fragments thereof, for example variants that incorporate one or more conservative amino acid substitutions.

The amino acid sequence for additional exemplary GHRH antagonists are provide:
5FPhAC-Ada-Tyr-DArg-Asp-Ala-Ile-5FPhe-Thr-Ala-Har-Tyr(Me)-Arg-Lys-Val-Leu-Abu-Gln-Leu-Ser-Ala-Arg-Lys-Leu-Leu-Gln-Asp-Ile-Nle-DArg-NHCH3 (AVR-235);
5FPhAC-Ada-Tyr-DArg-Asp-Ala-Ile-Cpa-Thr-Asn-Har-Tyr(Me)-Arg-Lys-Val-Leu-Abu-Gln-Leu-Ser-Ala-Arg-Lys-Leu-Leu-Gln-Asp-Ile-Nle-DArg-Har-Ada-NH2 (AVR-333);
5FPhAC-Ada-Tyr-DArg-Asp-Ala-Ile-5FPhe-Thr-Ala-Har-Tyr(Me)-Arg-Lys-Val-Leu-Abu-Gln-Leu-Ser-Ala-Arg-Lys-Leu-Leu-Gln-Asp-Ile-Nle-DArg-Har-Ada-NH2 (AVR-352);
5FPhAC-Ada-Tyr-DArg-Asp-Ala-Ile-Cpa-Thr-Ala-Har-5FPhe-Arg-Lys-Val-Leu-Abu-Gln-Leu-Ser-Ala-Arg-Lys-Leu-Leu-Gln-Asp-Ile-Nle-DArg-Har-Ada-NH2 (AVR-353);
5FPhAC-Ada-Tyr-DArg-Asp-Ala-Ile-Cpa-Thr-Ala-Har-5FPhe-Arg-Lys-Val-Leu-Abu-Gln-Leu-Ser-Ala-Arg-Lys-Leu-Leu-Gln-Asp-Ile-Nle-DArg-Har-NHCH3 (AVR-354); PhAC-Ada-Tyr-DArg-Asp-Ala-Ile-Cpa-Thr-Ala-Arg-Tyr(Me)-Arg-Lys-Val-Leu-Abu-Gln-Leu-Ser-Ala-His-Om-Leu-Leu-Gln-Asp-Ile-Nle-DArg-Har-NH2 (AVR-104);
PhAC-Ada-Tyr-DArg-Asp-Ala-Ile-Cpa-Thr-Ala-Arg-Tyr-Arg-Lys-Val-Leu-Abu-Gln-Leu-Ser-Ala-Arg-Lys-Leu-Leu-Gln-Asp-Ile-Nle-DArg-Har-NH2 (AVR-107);
PhAC-Ada-Tyr-DArg-Asp-Ala-Ile-Cpa-Thr-Ala-Arg-5FPhe-Arg-Lys-Val-Leu-Abu-Gln-Leu-Ser-Ala-His-Orn-Leu-Leu-Gln-Asp-Ile-Nle-DArg-Har-NH2 (AVR-116);
D-Phe-Ada-Tyr-DArg-Asp-Ala-Ile-Cpa-Thr-Ala-Arg-Tyr(Me)-Arg-Lys-Val-Leu-Abu-Gln-Leu-Ser-Ala-His-Orn-Leu-Leu-Gln-Asp-Ile-Nle-DArg-Har-NH2 (AVR-120);
PhAC-Ada-Tyr-DArg-Asp-Ala-Ile-Cpa-Thr-Ala-Arg-Amp-Arg-Lys-Val-Leu-Abu-Gln-Leu-Ser-Ala-His-Orn-Leu-Leu-Gln-Asp-Ile-Nle-DArg-Har-NHCH3 (AVR-20);
PhAC-Ada-Tyr-DArg-Asp-Ala-Ile-Cpa-Thr-Asn-Arg-Tyr(Me)-Arg-Lys-Val-Leu-Abu-Gln-Leu-Ser-Ala-Arg-Lys-Leu-Leu-Gln-Asp-Ile-Nle-DArg-Har-NHCH3 (AVR-234);
PhAC-Ada-Tyr-DArg-Asp-Ala-Ile-Cpa-Thr-Asn-Har-Tyr(Me)-Arg-Lys-Val-Leu-Abu-Gln-Leu-Ser-Ala-Arg-Lys-Leu-Leu-Gln-Asp-Ile-Nle-DArg-Har-Aoc-NHCH3 (AVR-321);
5FPhAC-Ada-Tyr-DArg-Asp-Ala-Ile-Cpa-Thr-Asn-Har-Tyr(Me)-Arg-Lys-Val-Leu-Abu-Gln-Leu-Ser-Ala-Arg-Lys-Leu-Leu-Gln-Asp-Ile-Nle-DArg-Har-Aoc-NHCH3 (AVR-322);
5FPhAC-Ada-Tyr-DArg-Asp-Ala-Ile-Cpa-Thr-Ala-Har-5FPhe-His-Om-Val-Leu-Abu-Gln-Leu-Ser-Ala-His-Orn-Leu-Leu-Gln-Asp-Ile-Nle-DArg-Har-NHCH3 (AVR-542);
5FPhAC-Ada-Tyr-DArg-Asp-Ala-Ile-Cpa-Thr-Ala-Har-5FPhe-His-Om-Val-Leu-Abu-Gln-Leu-Ser-Ala-His-Orn-Leu-Leu-Gln-Asp-Ile-Nle-DArg-Har-Ada-NHCH3 (AVR-543);
5FPhAC-Ada-Tyr-DArg-Asp-Ala-Ile-Cpa-Thr-Ala-Har-5FPhe-His-Om-Val-Leu-Abu-Gln-Leu-Ser-Ala-His-Orn-Leu-Leu-Gln-Asp-Ile-Nle-DArg-Har-Ada-NH2 (AVR-552);
5FPhAC-Ada-Tyr-DArg-Asp-Ala-Ile-5FPhe-Thr-Ala-Har-Tyr(Me)-His-Orn-Val-Leu-Abu-Gln-Leu-Ser-Ala-His-Om-Leu-Leu-Gln-Asp-Ile-Nle-DArg-Har-Ada-NH2 (AVR-553); 5FPhAC-Ada-Tyr-DArg-Asp-Ala-Ile-Cpa-Thr-Asn-Arg-Tyr(Me)-Arg-Lys-Val-Leu-Abu-Gln-Leu-Ser-Ala-His-Om-Leu-Leu-Gln-Asp-Ile-Nle-DArg-Har-Ada-NH2 (AVR-620) and combinations thereof.

As used herein, the term “peptide” refers to a linear molecule formed by binding amino acid residues to each other via peptide bonds. As used herein, the term “polypeptide” refers to a polymer of (the same or different) amino acids bound to each other via peptide bonds.

Analogs, fragments and variants of GHRH and any of the growth hormone releasing hormone peptides described herein can be synthesized using standard techniques of peptide chemistry.

In some aspects, the growth hormone releasing hormone peptides described herein can be further modified to improve stability. In some aspects, growth hormone releasing hormone peptides can have at least one amino acid residue that has an acetyl group, a fluorenylmethoxy carbonyl group, a formyl group, a palmitoyl group, a myristyl group, a stearyl group, or polyethylene glycol. In some aspects, an acetyl protective group can be bound to the growth hormone releasing hormone peptides described herein.

As used herein, the term “stability” refers to storage stability (e.g., room-temperature stability) as well as in vivo stability. The foregoing protective group can protect the peptides described herein from the attack of protein cleavage enzymes in vivo.

As used herein, the term “growth hormone releasing hormone peptide” can also be used to include functional equivalents of the growth hormone releasing hormone peptides described herein or variants thereof. As used herein, the term “functional equivalents” can refer to amino acid sequence variants having an amino acid substitution, addition, or deletion in some of the amino acid sequence of the growth hormone releasing hormone peptides while simultaneously having similar or improved biological activity, compared with the growth hormone releasing hormone peptides as described herein. In some aspects, the amino acid substitution can be a conservative substitution. Examples of the naturally occurring amino acid conservative substitution include, for example, aliphatic amino acids (Gly, Ala, and Pro), hydrophobic amino acids (Ile, Leu, and Val), aromatic amino acids (Phe, Tyr, and Trp), acidic amino acids (Asp and Glu), basic amino acids (His, Lys, Arg, Gln, and Asn), and sulfur-containing amino acids (Cys and Met).

Any of the compositions disclosed herein can further comprise a pharmaceutically acceptable carrier. In some aspects, the pharmaceutically acceptable carrier for the growth hormone releasing hormone peptides can be buffered saline. In some aspects, the pharmaceutically acceptable carrier for the small molecule can be water or DMSO. In some aspects, the pharmaceutically acceptable carrier can comprise a lipid-based or polymer-based colloid. In some aspects, the colloid can be a liposome, a hydrogel, a micro particle, a nanoparticle, or a block copolymer micelle.

In some embodiments, the therapeutically effective amount of the GHRH antagonist is administered intravenously, intramuscularly, subcutaneously or orally.

### III. Pharmaceutical Formulations

In some embodiments, the GHRH antagonists are formulated into a pharmaceutical composition. In some embodiments, the growth hormone releasing hormone (GHRH) antagonist are MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, and AVR-620. The disclosed pharmaceutical compositions can be for formulated for administration by parenteral (intramuscular, intraperitoneal, intravenous or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration. The compositions can be administered systemically or locally.

In one embodiment, the GHRH antagonists can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g., as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

In some embodiments, the disclosed formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6^{th} Edition, Ansel et. al., (Media, PA: Williams and Wilkins, 1995) which provides information on carriers, materials, equipment and process for preparing tablets and capsules and delayed release dosage forms of tablets, capsules, and granules.

In one embodiment, the growth hormone releasing hormone (GHRH) antagonists' can be administered to a subject with or without the aid of a delivery vehicle. Appropriate delivery vehicles are known in the art and can be selected to suit the particular active agent. For example, in some embodiments, the active agent(s) is/are incorporated into or encapsulated by, or bound to, a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

### IV. Methods of Administration

### A. Formulations for Parenteral Administration

In one embodiment, growth hormone releasing hormone (GHRH) antagonists' pharmaceutical compositions can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### B. Oral Immediate Release Formulations

Another embodiment provides suitable oral dosage forms containing of the growth hormone releasing hormone (GHRH) antagonists' that include but are not limited to tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

In some embodiments, binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

In some embodiments, lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

In some embodiments, stabilizers are used to inhibit or retard drug decomposition reactions which include, by way of example, oxidative reactions.

Some embodiments include surfactants. The surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

In some embodiments the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The agents can be chemically modified so that oral delivery of the derivative is efficacious. Generally, the chemical modification contemplated is the attachment of at least one moiety to the component molecule itself, where the moiety permits uptake into the blood stream from the stomach or intestine, or uptake directly into the intestinal mucosa. Also desired is the increase in overall stability of the component or components and increase in circulation time in the body. PEGylation is an exemplary chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The agent can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects.

For oral formulations, the location of release may be the stomach, the small intestine (the duodenum, the jejunum, or the ileum), or the large intestine. In some embodiments, the release will avoid the deleterious effects of the stomach environment, either by protection of the agent (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

Additionally, the capsule, pill, tablet, or syrup for oral administration should be stored in a manner so as to preserve its efficacy. Methods of storage include but are not limited to refrigeration, freezing, or storing at room temperature. If stored at room temperature, the probiotic should be stored in an air tight container.

### C. Extended Release Dosage Forms

One embodiment provides extended release formulations of growth hormone releasing hormone (GHRH) antagonists' that are generally prepared as diffusion or osmotic systems, for example, as described in “Remington—The science and practice of pharmacy” (20th ed., Lippincott Williams & Wilkins, Baltimore, MD, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations of the compounds of Formulas I and II can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray—congealed or congealed and screened and processed.

### D. Delayed Release Dosage Forms

In some embodiments delayed release formulations of growth hormone releasing hormone (GHRH) antagonists are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### E. Formulations for Mucosal and Pulmonary Administration

The growth hormone releasing hormone (GHRH) antagonists' pharmaceutical compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the growth hormone releasing hormone (GHRH) antagonists are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

One embodiment provides for nasal delivery for administration of the growth hormone releasing hormone (GHRH) antagonists'.

The growth hormone releasing hormone (GHRH) antagonists' can be formulated as an aerosol. The term aerosol refers to any preparation of a fine mist of particles, which can be in solution or a suspension, whether or not it is produced using a propellant. Aerosols can be produced using standard techniques, such as ultra-sonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. non-aqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

### F. Topical and Transdermal Formulations

Transdermal formulations containing the growth hormone releasing hormone (GHRH) antagonists' may also be prepared. These will typically be gels, ointments, lotions, sprays, or patches, all of which can be prepared using standard technology. Transdermal formulations can include penetration enhancers.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ© 76 (stearyl poly(10 oxyethylene ether), BRIJ© 78 (stearyl poly(20)oxyethylene ether), BRIJ© 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

In some embodiments, the composition is formulated for transdermal delivery and administered using a transdermal patch. In some embodiments, the formulation, the patch, or both are designed for extended release of the growth hormone releasing hormone (GHRH) antagonists'.

Exemplary symptoms, pharmacologic, and physiologic effects are discussed in more detail below.

### G. Methods of Manufacture

As will be appreciated by those skilled in the art and as described in the pertinent texts and literature, a number of methods are available for preparing formulations containing the growth hormone releasing hormone (GHRH) antagonists' including but not limited to tablets, beads, granules, microparticle, or nanparticles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

An exemplary method for preparing extended release tablets includes compressing a drug-containing blend, e.g., blend of drug-containing granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### V. Methods of Use

In some embodiments, the disclosed GHRH antagonist compositions can be used, for example, to treat or inhibit diabetes, to improve glycemic status, and to inhibit or treat weight gain, and to reduce triglyceride levels by administering to a subject in need thereof an effective amount of a GHRH antagonist to treat or inhibit diabetes, to improve glycemic status, and to inhibit or treat weight gain, and to reduce triglyceride levels.

In some embodiments, the effect of the composition on a subject is compared to a control. For example, the effect of the composition on a particular symptom, pharmacologic, or physiologic indicator can be compared to an untreated subject or the condition of the subject prior to treatment. In some embodiments, the symptom, pharmacologic, or physiologic indicator is measured in a subject prior to treatment, and again one or more times after treatment is initiated. In some embodiments, the control is a reference level, or an average determined from measuring the symptom, pharmacologic, or physiologic indicator in one or more subjects that do not have the disease or condition to be treated (for example, healthy subjects). In some embodiments, the effect of the treatment is compared to a conventional treatment that is known in the art.

### A. Regulation of Glucose Metabolism

Methods of using the disclosed compositions to improve glycemic status are disclosed. Methods typically include administering a subject in need thereof an effective amount of a GHRH antagonist composition.

### 1. Diabetes

The disclosed GHRH antagonist compositions can be used, for example, to treat or prevent diabetes in subjects in need thereof. Diabetes is a wide-spread disease affecting subjects of all ages. In some embodiments, the GHRH antagonist compositions are formulated at a dose to treat adults. In other embodiments, the GHRH antagonist compositions are formulated at a dose that is safe to treat children.

The disclosed GHRH antagonist compositions and methods of use thereof can be used to treat pre-diabetes, diabetes type I, or diabetes type II. In some embodiments the disclosed methods can be used to prophylactically or therapeutically alleviate, reduce, or inhibit one or more symptoms or physiological aspects of diabetes. Diabetes can be inhibited or reduced in a subject by administering to the subject an effective amount of the disclosed compositions.

Prediabetes is diagnosed if a subject has an A1C level between 5.7% and 6.4%. In one embodiment, administration of the growth hormone releasing hormone (GHRH) antagonists' composition is used to reduce levels of blood A1C and improve glycemic index. In one embodiment, administration of the growth hormone releasing hormone (GHRH) antagonists' composition can reduce A1C levels to below 5.7%.

Type-2 diabetes occurs because of impaired pancreatic insulin secretion coupled with decreased insulin sensitivity by target cells, leading to insulin resistance with chronic and persistent hyperglycemia. Peripheral insulin resistance occurs because of impaired insulin-induced signal transduction that normally causes membrane translocation of glucose transporters such as GLUT4 from the cytosol. In one embodiment, administration of the GHRH antagonist composition is used to reduce levels of blood A1c and improve glycemic index. In another embodiment, administration of the GHRH antagonist composition is used to eliminate symptoms and co-morbidities of diabetes including weight gain.

### 2. Treatment of Obesity

Methods of using the disclosed GHRH antagonist compositions to treat or prevent weight gain in a subject in need thereof are provided. Methods include administering to a subject in need thereof an effective amount of a GHRH antagonist composition. One embodiment provides administering an effective amount of a GHRH antagonist composition to treat or prevent weight gain.

### B. Co-Therapies

In one embodiment, the disclosed probiotic compositions can be administered to a subject in need thereof in combination with a second therapeutic agent including, but not limited to insulin, metformin, appetite suppressors, other diabetes medication, anti-inflammatory agents, or combinations thereof. In some embodiments, the disclosed GHRH antagonist compositions and the additional therapeutic agent are administered separately, but simultaneously, or in combination or alternation. The disclosed compositions and the additional therapeutic agent can also be administered as part of the same composition. In other embodiments, the disclosed compositions and the second therapeutic agent are administered separately and at different times, but as part of the same treatment regime.

### 1. Treatment Regimens

The subject can be administered a first therapeutic agent 1, 2, 3, 4, 5, 6, or more hours, or 1, 2, 3, 4, 5, 6, 7, or more days before administration of a second therapeutic agent. In some embodiments, the subject can be administered one or more doses of the first agent every 1, 2, 3, 4, 5, 6 7, 14, 21, 28, 35, or 48 days prior to a first administration of second agent. The disclosed compositions can be the first or the second therapeutic agent.

The disclosed GHRH antagonist compositions and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

In some embodiments, the disclosed growth hormone releasing hormone (GHRH) antagonists' compositions are combined with or co-administered with diabetes medication, including sulfonylureas, biguanides, meglitinides, thazolidinediones, DPP-4 inhibitors, SGLT2 inhibitors, alpha-glucosidase inhibitors, and bile acid sequestrants. Exemplary sulfonylureas include but are not limited to chlorpropamide, glipizide, glyburide, and glimepiride. Exemplary biguanides include metformin. Exemplary meglitinides include but are not limited to repaglinide and nateglinide. Exemplary thiazolidinediones include but are not limited to, rosiglitazone and pioglitazone. Exemplary DPP-4 inhibitors include but are not limited to, sitagliptin, saxaliptin, linagliptin, and alogliptin. Exemplary SGLT2 inhibitors include but are not limited to, canagliflozin and dapagliflozin. Exemplary bile acid sequestrants include but are not limited to, colesevelam, colesevelam hydrochloride, cholestyramine, and colestimide.

### VI. Kits

The GHRH antagonist compositions can be supplied in pill or tablet form with the quantity indicated.

In an alternative embodiment, the GHRH antagonist can be supplied in liquid form in a hermetically sealed container indicating the quantity and concentration. Pharmaceutical packs and kits including one or more containers filled with a GHRH antagonist are also provided. Additionally, one or more other prophylactic or therapeutic agents useful for the treatment of a disease can also be included in the pharmaceutical pack or kit. Optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLES

### Example 1: Evaluate the pGHRH-R and GHRH Tissue Levels in Normal Wild Type C57BL/6 Male Mice

### Materials and Methods

### Animals:

Peripheral growth hormone releasing hormone receptor (pGHRH-R) and growth hormone releasing hormone (GHRH) tissue levels in protein extracts from duodenum, jejunum-ileum, and liver in wild type C57BL/6 male mice were measured by ELISA.

### Results

Recent studies highlight the role of the intestine in whole body lipid homeostasis. Previously published data demonstrate a novel role for peripheral Growth Hormone Releasing Hormone Receptors (pGHRH-R) in a rodent model of type 1 diabetes (T1D)-associated dyslipidemia. Expression of pGHRH-R is increased in the small intestine of rats injected with streptozotocin (STZ) to induce T1D. In addition, specific activation of pGHRH-R with the GHRH agonist MR-409, promotes secretion of apolipoprotein B48 (ApoB-48), a component of chylomicrons, in rat small intestine epithelial cells (IEC) loaded in vitro with oleic acid, a fatty acid that is naturally present in various animal and vegetable fats and oils. This effect is associated with dysfunction of the receptor for the incretin Glucagon-like peptide-1 (GLP-1R), which is expressed in the intestine and reduces TRL levels in vivo. Administration of the GHRH antagonist MIA-602 reduced ApoB-48 lipoproteins both in STZ-rats and in rat IEC in vitro (Patent #U.S. Pat. No. 10,201,588 B2).

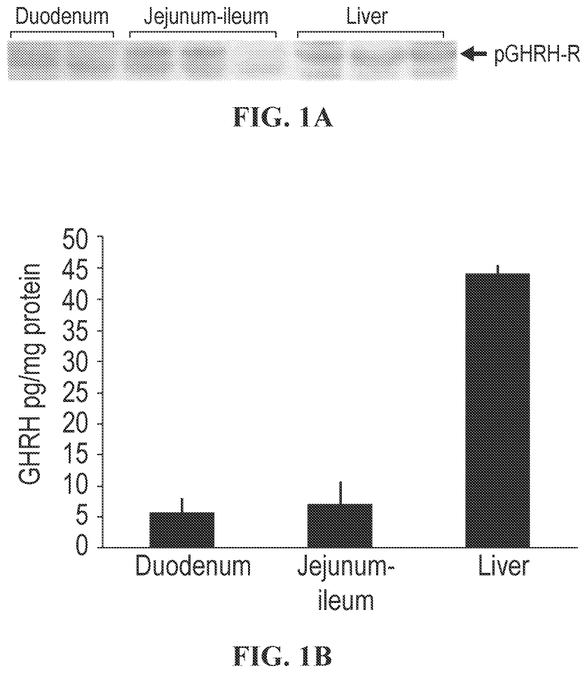

As such, the data presented herein indicates that peripheral GHRH-R is expressed not only in small intestine of rats but also in the liver and the small intestine of normal C57BL6 mice, where GHRH ligand is also present. FIGS. 1A and 1B show peripheral growth hormone releasing hormone receptor (pGHRH-R) and GHRH tissue levels in normal wild type C57BL/6 male mice. FIG. 1A is a Western blot of mouse duodenum, jejunum-ileum, and liver extracts showing expression of pGRH-R. FIG. 1B is a bar graph showing growth hormone releasing hormone (GHRH) (pg/mg protein) and the ligand of pGHRH-R from mouse duodenum, jejunum-ileum, and liver extracts measured by ELISA (MyBioSource).

### Example 2: Evaluate the Effect of the Treatment of GHRH Antagonist MIA-602 on Total Triglycerides, ApoB-48, and VLDL Levels in db/db Mice

### Materials and Methods

### Animals:

Obese 20-week old male diabetic (db/db) mice were treated with the GHRH antagonist MIA-602 (25 μg/kg/day, subcutaneous, every other day) or vehicle (untreated, n=6/group) for 4 weeks. Total triglycerides (mg/dl), ApoB-48-containing lipoproteins (ApoB-48, ng/ml), and very low density lipoprotein (VLDL, mg/dl) in plasma obtained from mice in the fasting state were measured by ELISA (MyBioSource).

### Results

Given the high incidence of T2D and obesity and its association with CVD, experiments were carried out to investigate whether GHRH receptor antagonist MIA-602 administered subcutaneous suppresses elevation of TRL in mice homozygous for the diabetes spontaneous mutation Leprdb-BKS db (db/db), a valid model of human T2D/obesity. Heterozygous for Leprdb were used as lean controls.

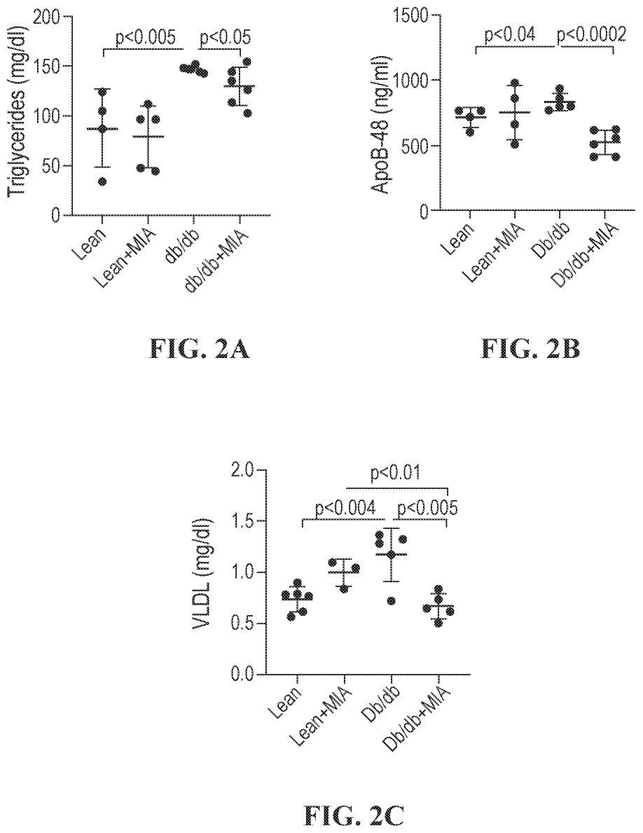

The db/db mice showed significant higher levels of triglycerides, ApoB-48, and VLDL in the fasting state, after 24 weeks of age as compared to lean control mice (FIGS. 2A-2C). GHRH antagonist MIA-602 (25 μg/kg/day, subcutaneous, every other day), was given to a sub-group of 20 weeks old db/db mice for 4 weeks. FIGS. 2A-2C show the effect of the treatment with MIA-602 on triglycerides, ApoB-48, and very low density lipoprotein (VLDL) levels, respectively, in db/db mice. FIG. 2A shows a significant increase of total triglycerides (mg/dl) in untreated db/db mice versus MIA-602 treated db/db mice. FIG. 2B shows a significant increase of total ApoB-48 (ng/ml) in untreated db/db mice versus MIA-602 treated db/db mice. FIG. 2C shows a significant increase of total VLDL (mg/dl) in untreated db/db mice versus MIA-602 treated db/db mice. Treatment with MIA-602 significantly reduced all lipid abnormalities in db/db mice as compared untreated db/db mice.

### Example 3: Evaluate the Microsomal Triglyceride Transfer Protein (MTTP) Activity and ApoB-48 Protein Expression in db/db Mice

### Materials and Methods

### Animals:

Protein extracts were isolated from jejunum of diabetic (db/db) mice and lean control mice. Microsomal triglyceride transfer protein (MTTP) activity (pmole/μg protein/hour) was measured in protein extracts from diabetic (db/db) mice versus lean control mice treated or not with GHRH antagonist MIA-602. Further, ApoB-48 protein expression determined by densitometric analysis (optical density, O.D.) of ApoB-48 corrected by protein loading (vinculin) was also performed in protein extracts from diabetic (db/db) mice versus lean control mice treated or not with GHRH antagonist MIA-602.

### Results

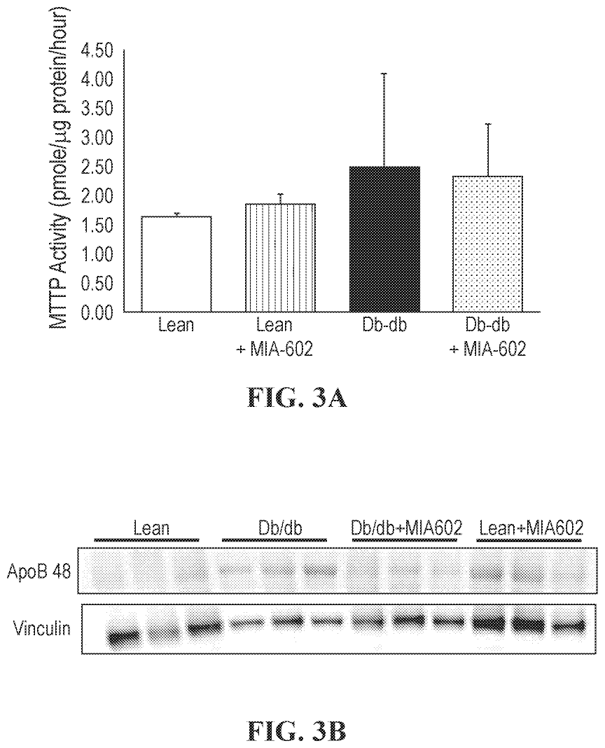

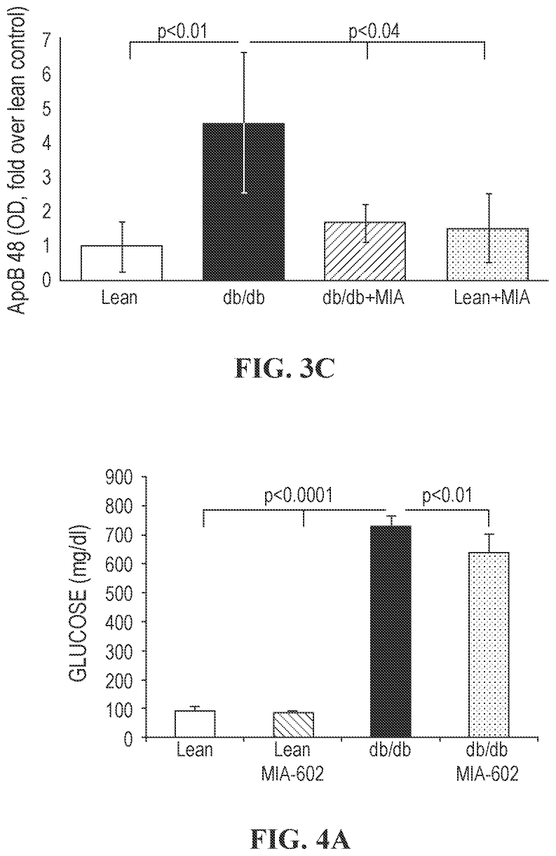

FIGS. 3A, 3B and 3C show microsomal triglyceride transfer protein (MTTP) activity and ApoB-48 levels in protein extracts from the jejunum of db/db mice treated with the GHRH antagonist MIA-602. The lowering effect of GHRH antagonist MIA-602 on plasma lipid levels observed in db/db mice, was not associated with significant changes in the activity of microsomal triglyceride transfer protein (MTTP) at least in the small intestine (FIG. 3A). However, significantly lower levels of ApoB-48 were found in protein extracts from the jejunum of db/db mice treated with the GHRH antagonist MIA-602, as compared to untreated animals (FIG. 3B-3C). It also shows that GHRH antagonist MIA-602 (25 μg/kg/day, s.c.) significantly reduced ApoB-48 content in small intestine of db/db mice as compared to untreated db/db mice.

### Example 4: Evaluate the Effect of GHRH Antagonist MIA-602 on Fasting Blood Glucose, Hemoglobin A1c (HbA1c), and Glucagon Levels in db/db Mice

### Materials and Methods

### Animals:

Male db/db mice, 12 weeks of age were treated with the GHRH antagonist MIA-602 (25 μg/kg/day, subcutaneous, n=6) or vehicle (untreated, n=6/group) for 12 weeks. Samples were obtained from tail bleeding to determine (A) Fasting blood glucose (AlphaTRAK), (B) HbA1c (Crystal Chem) and (C) Glucagon.

### Results

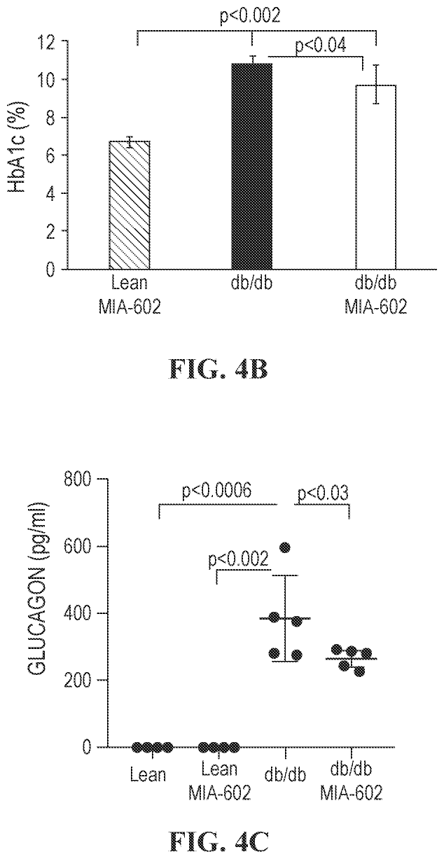

FIGS. 4A-4C show the effect of MIA-602 on fasting blood glucose, Hemoglobin A1c (HbA1c), and glucagon levels in db/db mice. FIG. 4A showed a significant increase of glucose level (mg/dl) in all db/db mice groups versus lean control. However, a modest but significant reduction in glucose level was seen in db/db mice treated with MIA-602 as compared to untreated db/db mice. FIG. 4B is a bar graph showing significant increase of hemoglobin A1c (HbA1c) (%) level in all db/db mice groups versus lean control. However, a modest but significant reduction in hemoglobin A1c (HbA1c) level was seen in db/db mice treated with MIA-602 as compared to untreated db/db mice. These results could be due to the observed reduction in glucagon levels observed in db/db mice treated with MIA-602, as compared to untreated db/db mice (FIG. 4C).

### Example 5: Diet-Induced Obesity (DIO) in Mouse Model: Body Weight and Blood Glucose Level

### Materials and Methods

### Animals:

Body weight (g) and blood glucose (mg/dl) in male C57BL/6 mice either on high fat diet (HFD) or on control diet (CD) for 12 weeks were evaluated.

### Results

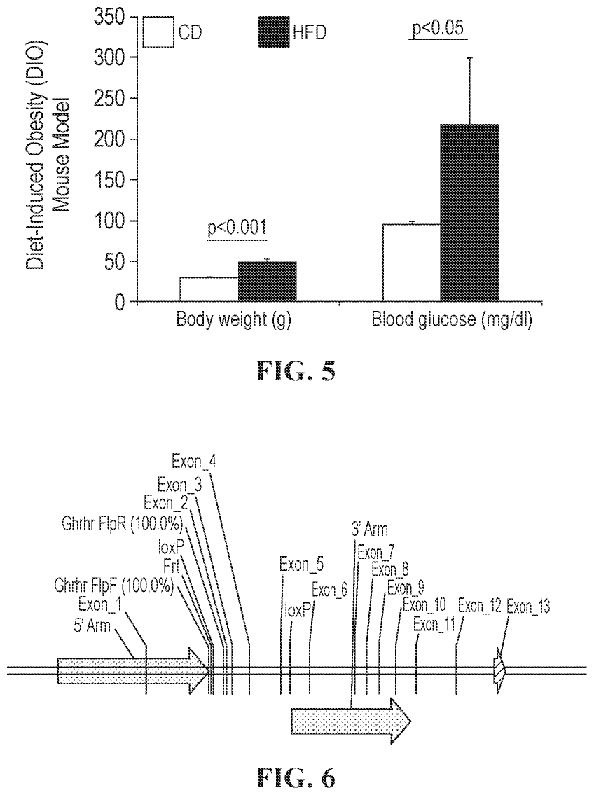

The increase of energy intake associated with a western diet is also a crucial determinant for dyslipidemia in obesity and the metabolic syndrome linked to T2D. Therefore, it was investigated whether the peripheral receptor of GHRH expressed in the epithelium of the small intestine plays a role in dyslipidemia in a mouse model of diet-induced obesity when mice were fed a high fat-supplemented diet (HFD). For the purpose of this study, 12 weeks of high fat diet was determined as the time point to initiate treatment with the newly developed highly potent GHRH antagonist AVR-352, structurally related to MIA-602. The reason is that after 12 weeks of high fat diet (HFD), mice had significant increase in body weight and also enhanced and fluctuated blood glucose levels in the fasting state (FIG. 5).

FIG. 5 shows at diet-induced obesity (DIO) in mouse model exhibiting mice fed with high fat diet (HFD) for 12 weeks versus mice on control diet (CD). Mice fed on HFD demonstrated significant increase in body weight, and enhanced as well as fluctuating blood glucose levels in the fasting state as compared to mice fed on CD. Values are mean±SD, n=5/CD; n=10/HFD. In addition, these mice exhibited some metabolic profile of T2D at this time point, as they were hyperinsulinemic and hyperleptinemic (Table 1).

### Example 6: Diet-Induced Obesity (DIO) in Mouse Model: Hormone Levels

### Materials and Methods

### Animals:

Hormone levels, insulin and leptin, in male C57BL/6 mice either on high fat diet (HFD) or on control diet (CD) for 12 weeks were evaluated. Plasma samples assayed with Mouse Metabolic Bead Panel (EMD Millipore).

### Results

Mice fed on high fat diet (HFD) exhibited some metabolic profile of T2D at this time point, as they were hyperinsulinemic and hyperleptinemic after 12 weeks of diet as compared to mice fed on control diet (CD). Table 1 shows insulin and leptin, hormone levels in male C57BL/6 mice either on high fat diet (HFD) or on control diet (CD) for 12 weeks.

TABLE 1
Diet-Induced Obesity (DIO) Mouse Model
Hormone levels after 12 wks of diets in C57BL/6 mice

| Diet | Insulin | Leptin |
|---|---|---|
| Control diet (CD) | <69* | 2220.4 ± 846.4 |
| High Fat diet (HFD) | 3564.3 ± 807.9 | 13084.9 ± 759.8′ |

### Example 7: Evaluate the Effect of Selective Deletion of the Ghrhr Gene in the Small Intestine

### Materials and Methods

### Animals:

Both 8-week old wild type mice with intact ghrhr gene, and ghrhr conditional knock out mice were fed a high fat or control diet for 16 weeks. The effect of selective deletion of the ghrhr gene in the small intestine versus the effect of the GHRH antagonist AVR-352 or vehicle treatment for the last 4 weeks in wild type mice when both groups were fed a high fat diet, on fasting and postprandial triglyceride plasma levels were compared. The Ghrhr floxed mice using embryonic ES Cells was generated (International Mouse Phenotyping Consortium—IMPC). The ES cell clones were targeted with a floxed allele of Ghrhr with exons 2-5 flanked by loxP sites, and were injected into C57BL/6-Albino blastocysts for chimera production and generation of Ghrhr flox/wt mice (Animal Models Core, UNC, Project ID: Ex231). The colony was expanded and obtained the homozygous Ghrhr flox/flox mice, which were cross bred with mice expressing Cre recombinase in the small intestine (B6.Cg-Tg(Vil1-cre) 1000 Gum/J, Jackson Laboratories). Recombinase activity results in the deletion of loxP flanked targets in the intestine.

### Results

A ghrhr conditional knock out mouse in a C57BL/6 background was generated. These mice lack the receptor for GHRH, as well as its bioactive splice variant SV-1 (Xiong X, et al., Proc Natl Acad Sci USA. 117(12):6726-6732 (2020)) exclusively in the intestinal epithelium (FIG. 6). Both 8-week old wild type mice with intact ghrhr gene, and ghrhr conditional knock out mice were fed a high fat or control diet for 16 weeks. The effect of selective deletion of the ghrhr gene in the small intestine was compared to the effect of the GHRH antagonist AVR-352 or vehicle treatment for the last 4 weeks in wild type mice when both groups were fed a high fat diet, on fasting and postprandial triglyceride plasma levels.

FIG. 6 exhibits full length Ghrhr gene map that comprises nucleotide sequence from exon 1 to exon 13. The splice variants (SV) of Ghrhr gene lack the first 3 exons. The bioactive SV-1, which has ligand-dependent but also independent activity, has the highest homology to the full receptor. SV1 has an intronic sequence (intron 3) corresponding to the translation initiation site followed by a nucleotide sequence derived from exon 4-13.

### Example 8: Evaluate the Effect of Expression of the GHRH Receptor in the Small Intestine Epithelial Cells of the Wild Type Mice

### Materials and Methods

### Animals:

Paraffin sections of small intestine were processed for immunostaining of the receptor for GHRH (Abcam). Detection was carried out using VECTASTAIN Elite ABC-HRP Kit, and the slides were developed with DAB (Vector) and counterstained with HE. Images were recorded using a confocal microscope.

### Results

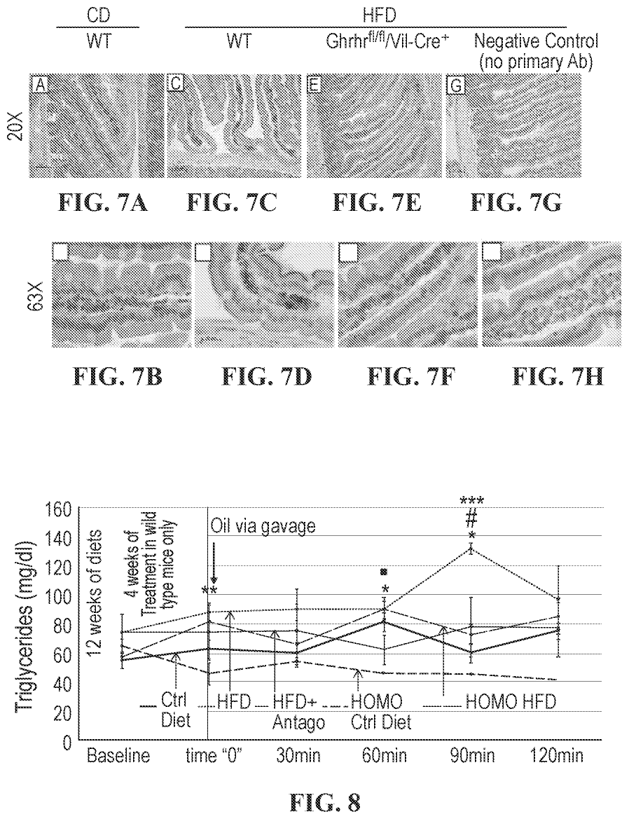

FIGS. 7A-7H show micrographs of paraffin sections of small intestine processed for immunostaining of the receptor for GHRH expression of wild type (WT) mice fed on control diet (CD) at 20× magnification (FIG. 7A) and at 63× magnification (FIG. 7B), WT mice fed on high fat diet (HFD) at 20× magnification (FIG. 7C) and at 63× magnification (FIG. 7D), WT mice fed on high fat diet (HFD) with a genetic ablation of the receptor of GHRH in the epithelium of small intestine (Ghrhr/Vil-Cre⁺) at 20× magnification (FIG. 7E) and at 63× magnification (FIG. 7F), and WT mice fed on high fat diet (HFD) with negative control (no primary Ab) at 20× magnification (FIG. 7G) and at 63× magnification (FIG. 7H), respectively. The expression of the GHRH receptor was increased in the small intestine epithelial cells of the wild type mice fed on high fat diet (HFD), as compared to the mice fed on control diet (CD). This effect was not observed in mice with a genetic ablation of the receptor of GHRH in the epithelium of small intestine.

### Example 9: Postprandial Triglyceride Plasma Levels

### Materials and Methods

### Animals:

Mice with the deletion of GHRH receptor in small intestine epithelium (HOMO) were fed on high fat diet (HFD) or control diet (Ctrl diet) for 16 weeks. AVR-352 (25 μg/kg/day, subcutaneous, every other day), was given to wild type mice after 12 weeks of high fat diet (HFD) for 4 weeks.

### Results

FIG. 8 is a graph showing postprandial triglyceride plasma levels demonstrating the effect of GHRH antagonist AVR-352 (Antago) or deletion of GHRH receptor in small intestine epithelium (HOMO) of mice fed on high fat diet (HFD) or control diet (Ctrl diet) for 16 weeks. AVR-352 (25 μg/kg/day, subcutaneous, every other day), was given to wild type mice after 12 weeks of high fat diet (HFD) for 4 weeks. Wild type mice fed a high fat diet had increased levels of triglycerides in the postprandial state versus fasting up to 2 hours after oral lipid loading, when compared with mice under control diet. This effect was significantly blunted by treatment with the GHRH antagonist AVR-352. The contribution of the GHRH receptor on increased triglyceride output from the small intestine, was unequivocally validated in mice with selective deletion of ghrhr gene in the epithelium of the small intestine. When these mice were fed on HFD or CD, the triglyceride levels after oral lipid loading were significantly reduced when compared to wild type mice fed on the same diets.

### Example 10: Evaluate the Effect of GHRH Antagonist AVR-352 on Fasting Triglyceride Levels in Diet-Induced Obesity (DIO) in C57BL/6 Mice

### Materials and Methods

### Animals:

C57BL/6 mice, 12 weeks of age were fed on a normal control diet (CD) or diet supplemented with high levels of fat (HFD) for 12 weeks, before treatment with GHRH antagonist, AVR-352 (25 μg/kg/day, subcutaneous) or vehicle for 12 weeks. Total triglycerides in plasma (TG ELISA kit, MyBioSource) were measured.

### Results

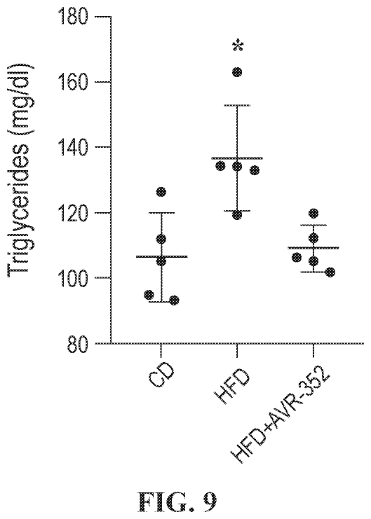

After wild type mice were fed a high fat diet for a longer period—24 weeks—plasma levels of triglycerides in the fasting state were significantly elevated when compared to mice fed a normal control diet. This effect was significantly blunted in high fat diet fed mice for 24 weeks that were treated with AVR-352 for the last 12 weeks. FIG. 9 shows effect of GHRH antagonist AVR-352 on fasting triglyceride levels in diet-induced obesity (DIO) in C57BL/6 mice. Total triglycerides measured in plasma showed significant higher levels in mice fed on high fat diet (HFD) as compared to mice fed on a normal control diet (CD) after 24 weeks. GHRH antagonist, AVR-352 significantly reduced triglycerides in HFD mice after 12 weeks of AVR-352 treatment when compared to untreated HFD mice.

### Example 11: Comprehensive Laboratory Animal Monitoring System (CLAMS): A) Food Intake, b) Body Weight Gain, c) Activity-Ambient, and d) Respiratory Exchange Ratio

### Materials and Methods

### Animals:

12-Week old C57BL/6 male mice were fed on a high fat diet (HFD) or control diet (CD) for 12-weeks. Mice fed on HFD were treated with the GHRH antagonist MIA-602 (25 μg/kg/day, subcutaneous) or vehicle for 12-weeks. At the end of treatment, mice were placed individually in metabolic (CLAMS) system and a) food intake, b) body weight gain, c) activity-ambient, and d) respiratory exchange ratio were measured.

### Results

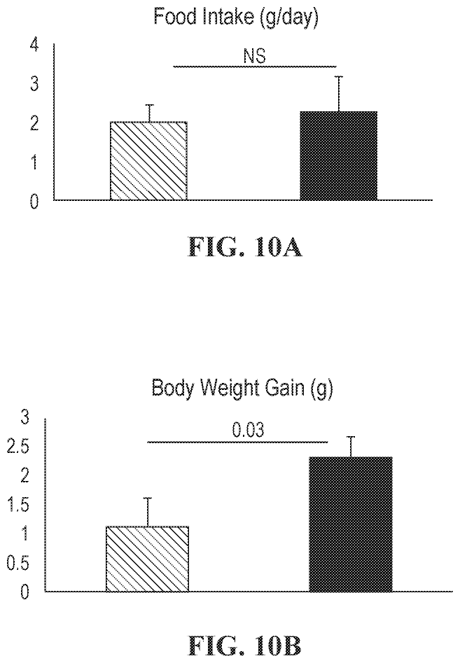

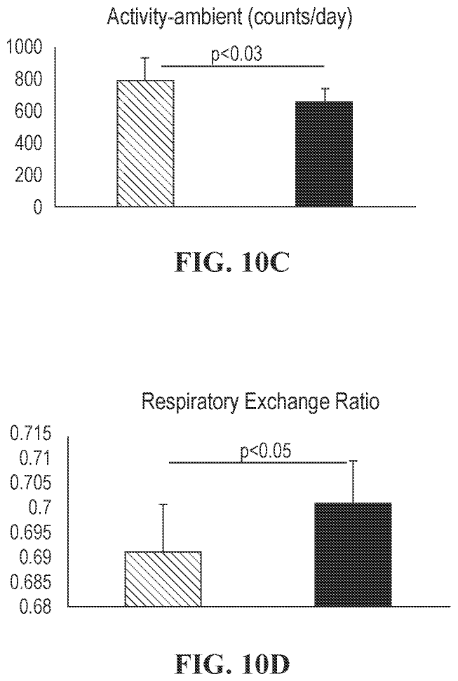

Comprehensive Lab Animal Monitoring System (CLAMS) was also used after 24 weeks of high fat diet and treatment with the GHRH antagonist MIA-602 or vehicle for the last 12 weeks of diet. No changes in food intake were observed between HFD fed mice treated or not with MIA-602 (FIG. 10A). However, MIA-602 induced a significant reduction in body weight gain (FIG. 10B), accompanied by a significant increase in daily activity-ambient (FIG. 10C), compared to untreated mice. Furthermore, the respiratory exchange ratio (RER) measured in obese mice after 24 weeks of high fat diet and treated with MIA-602 for the last 12 weeks of diet was slightly but significantly lower as compared to non-treated mice (FIG. 10D). The respiratory exchange ratio (RER) determines the muscle capacity of fatty acid oxidation. The inability of fatty acid utilization is associated with the accumulation of lipids in peripheral tissues, which contributes to insulin resistance in obesity.

### Example 12: Evaluate the Effect of GHRH Antagonist AVR-352 or Ablation of GHRH Receptor Gene (HOMO) on ApoB-48 and MTTP Content in Small Intestine of Mice

### Materials and Methods

### Animals:

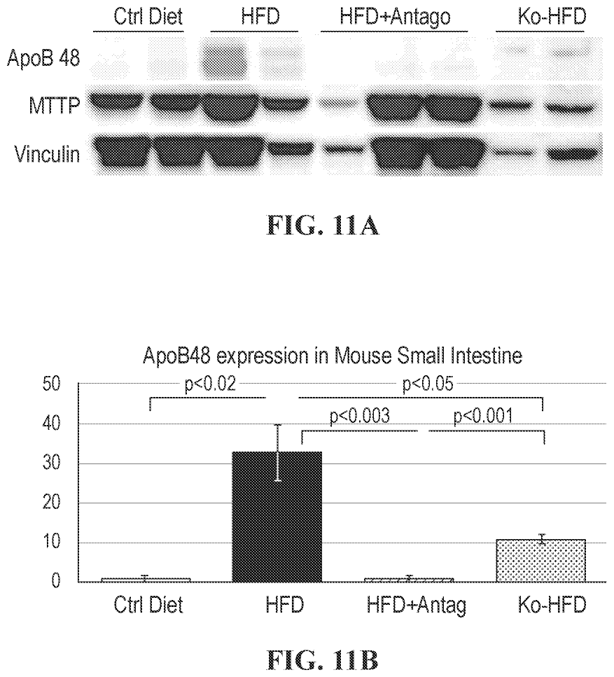

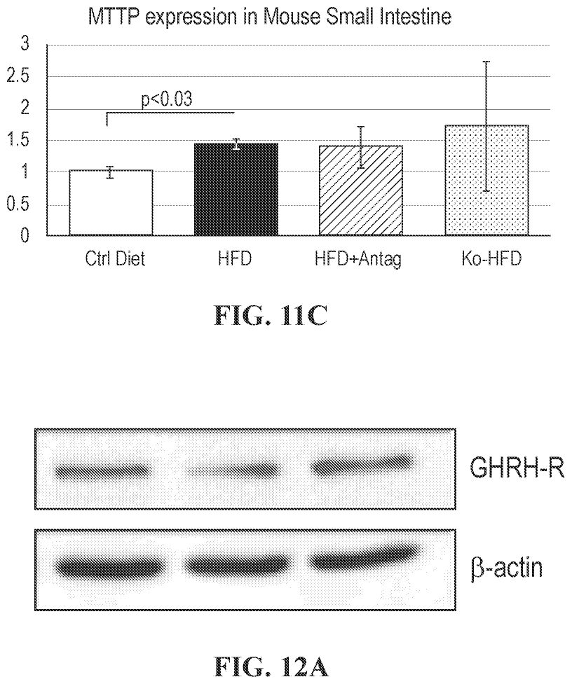

The protein expression of microsomal triglyceride transfer protein (MTTP), an enzyme involved in ApoB-48 lipoprotein assembly, in protein extracts isolated from scrapped epithelium of whole small intestine of mice were evaluated. Densitometric analysis (optical density O.D., fold over Ctrl diet fed mice) of ApoB-48 (FIG. 11B) and MTTP (FIG. 1C) corrected by protein loading (vinculin) (FIG. 11A) was performed. Mice fed on HFD were treated with GHRH antagonist AVR-352 (Antago, 25 μg/kg/day, subcutaneous, every other day for 4 weeks).

### Results

Evaluation of protein expression of microsomal triglyceride transfer protein (MTTP), an enzyme involved in ApoB-48 lipoprotein assembly, in protein extracts from scrapped epithelium of whole small intestine of mice showed a significant increase of MTTP in mice fed a high fat diet as compared to mice fed a control diet (FIG. 11A, 11C). This effect was accompanied by a significant increase of ApoB-48 expression, a component of chylomicrons, in untreated wild type mice fed a high fat diet. Treatment of wild type mice fed a high fat diet with AVR-352, induced a significant reduction of APOB-48, without significantly affecting MTTP protein (FIG. 11A, 11B). The ablation of the receptor of GHRH in the small intestine in Ghrhr conditional knock out mice, had similar effects on ApoB-48 and MTTP protein expression as in mice treated with AVR-352, when fed a high fat diet (FIG. 11A-11C).

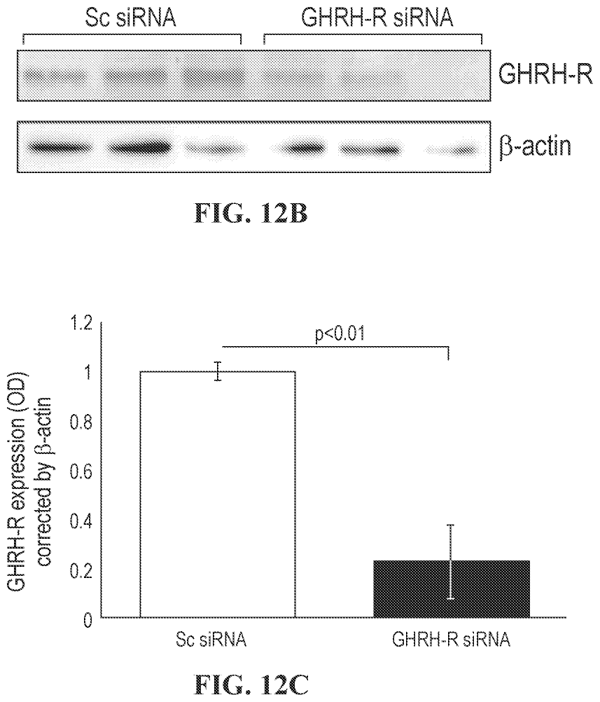

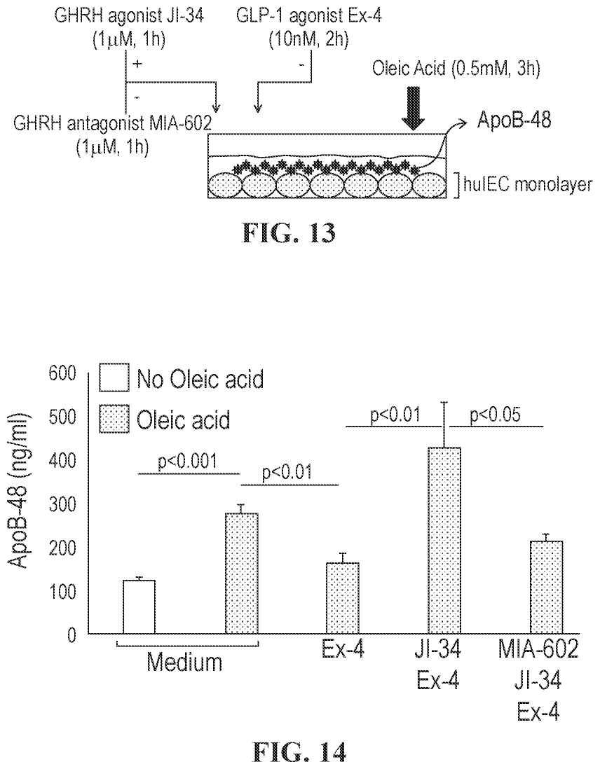

Human primary small intestine epithelial cells (huIEC, ATCC) also express the peripheral receptor for GHRH (FIG. 12A). As a translational aspect of the studies, it was also determined whether activation of the GHRH receptor with synthetic peptide agonists of growth hormone releasing hormone (GHRH) JI-34, MR-409 (Cai R, et al., Peptides, 52:104-12 (2014), or native GHRH promotes secretion of lipoproteins (LP) containing apolipoprotein B48 (ApoB-48), a marker of chylomicrons, in human primary small intestine epithelial cells (huIEC) upon loading with oleic acid in vitro (FIG. 13). huIEC were also depleted of GHRH receptor by siRNA transfection. Non-targeting (Sc) siRNA was used as control (FIG. 12B-12C).

### Example 13: Evaluate the Effect of GHRH Agonist JI-34, GLP-1 Receptor Agonist Exendin 4 (Ex-4) and GHRH Antagonist MIA-602 on ApoB-48 Levels in Confluent Monolayers of Human Small Intestine Epithelial Cells (hulECs)

### Materials and Methods

### Animals:

Confluent monolayers of human small intestine epithelial cells (hulECs, ATCC) were cultured in 6-well plates and were either loaded with oleic acid or not loaded with oleic acid after treatment. ApoB-48 was measured in culture supernatants (MyBioSource).

### Results

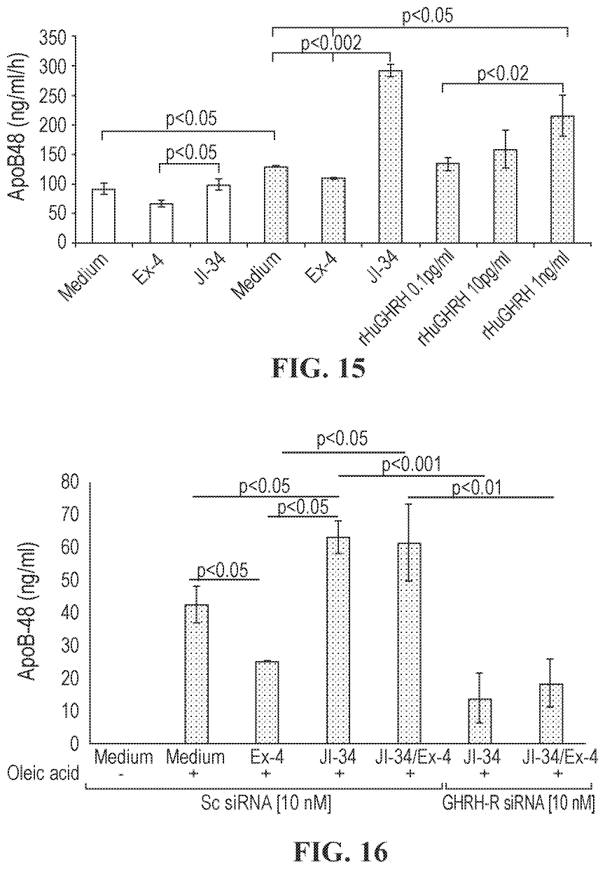

When huIEC were exposed to the GHRH agonist JI-34 (or MR-409), there was a significant elevation of ApoB-48 released in the culture medium when loaded with oleic acid, as compared with cells in medium or treated with the glucagon-like peptide-1 (GLP-1) agonist exendin-4 (Ex-4) alone (FIG. 14-15). GLP-1 receptor is known to reduce TRL-C levels in vivo, and these results are in agreement with data found in rat small intestine epithelial cells (IEC) loaded in vitro with oleic acid. The effect of JI-34 on enhanced ApoB-48 secretion by huIEC was maintained even when co-administered with Ex-4 (FIG. 14), indicating GHRH agonist also impaired the actions of Ex-4 on GLP-1 receptor function, similar to rodent cells. Moreover, upon exposure of huIEC to different concentrations of native growth hormone releasing hormone (GHRH), the naturally occurring ligand of GHRH receptor, increased levels of ApoB-48 were released in the culture medium in a dose-dependent manner (FIG. 15). Co-treatment of huIEC with the GHRH antagonist MIA-602, completely restored the impaired signaling of Ex-4 in the presence of JI-34 (FIG. 14).

### Example 14: Evaluate the Effect of GHRH Agonist JI-34 in Sc siRNA and GHRH-R siRNA on ApoB-48 Levels in Confluent Monolayers of Human Small Intestine Epithelial Cells (hulECs)

### Materials and Methods

### Animals:

Confluent monolayers of human small intestine epithelial cells (hulECs, ATCC) were cultured in 24-well plates and transfected for 96 hours with GHRH-R siRNA (10 nM, Dharmacon). As control siRNA, non-targeting scrambled (Sc) sequence was used for transfection (10 nM, Dharmacon). Cells were then either loaded with oleic acid or not loaded with oleic acid after treatments, and ApoB-48 was measured in culture supernatants (MyBioSource).

### Results

The actions of the GHRH agonist-GHRH receptor complex in huIEC are specific and not mediated by off-target effects. This was verified by depleting human IEC of GHRH receptor by siRNA transfection (FIG. 12B-12C). Control cells were transfected with non-targeting scrambled (Sc) siRNA (FIG. 12B-12C). GHRH agonist JI-34 increased ApoB-48-containing lipoproteins (LP) levels in non-targeting siRNA-transfected cells (FIG. 16), but these actions were completely abrogated in cells depleted of GHRH receptor upon transfection with GHRH receptor siRNA (FIG. 16).

### Example 15: Comprehensive Laboratory Animal Monitoring System (CLAMS): A) Body Weight Gain (%), and b) Activity-Ambient (Counts/Day)

### Materials and Methods

### Animals:

8 to 10-Week old C57BL/6 wild type male mice were fed on a high fat diet (HFD) for 12-weeks. Mice were then treated with the GHRH antagonist AVR-352 (25 μg/kg/day, subcutaneous) or vehicle for 4-weeks. At the end of treatment, mice were placed individually in metabolic (CLAMS) system. Body weight was recorded at beginning of treatment and at time of CLAMS after 4-weeks of treatment with the GHRH antagonist AVR-352 or vehicle. Activity-ambient (counts/day) was also measured.

### Results

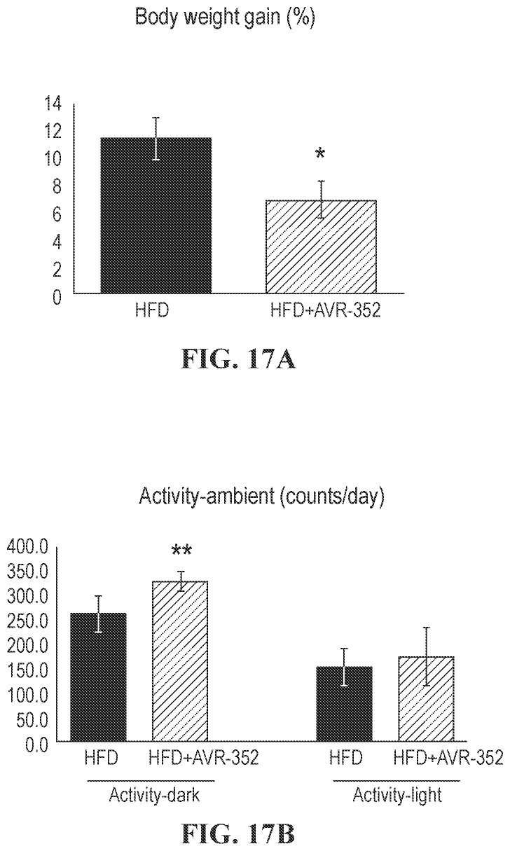

FIGS. 17A and 17B represent Comprehensive Laboratory Animal Monitoring System (CLAMS) showing a) body weight gain, and b) activity-ambient. FIG. 17A is a bar graph showing body weight gain (%) in AVR-352 treated HFD mice and HFD mice. AVR-352 treated HFD mice had significantly lower body weight gain than untreated HFD mice. FIG. 17B is a bar graph showing activity-ambient (counts/day) in AVR-352 treated HFD mice and HFD mice in activity-dark cycle and activity-light cycle. AVR-352 treated HFD mice had significantly higher daily activity-ambient than untreated HFD mice during the dark cycle. Further, no significant differences were seen during the light cycle.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of treating a metabolic disease or syndrome in a subject in need thereof comprising administering to the subject a therapeutically effective amount of a growth hormone releasing hormone (GHRH) antagonist or a pharmaceutical composition comprising the GHRH antagonist to reduce triglyceride-rich-lipoproteins (TRL) in the subject to treat the metabolic condition or syndrome.

2. The method of claim 1, wherein the metabolic disease or syndrome is pre-diabetes, type I diabetes, type II diabetes, gestation diabetes, obesity, or a combination thereof.

3. The method of claim 1 or 2, wherein the GHRH antagonist is selected from the group consisting of MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof.

4. The method of any one of claims 1-3, wherein the therapeutically effective amount of the GHRH antagonist is administered parenterally or enterally.

5. The method of any one claims 1-4, wherein the therapeutically effective amount of the GHRH antagonist is administered before a meal.

6. The method of any one of claims 1-4, wherein the therapeutically effective amount of the GHRH antagonist is administered before or after a meal.

7. The method of any one of claims 1-6, wherein the therapeutically effective amount of the GHRH antagonist is administered continuously or intermittently.

8. The method of claim 1, wherein the subject is a human.

9. A method of treating an obesity in a subject in need thereof, comprising administering to the subject a therapeutically effective amount of a growth hormone releasing hormone (GHRH) antagonist.

10. The method of claim 9, wherein the growth hormone releasing hormone (GHRH) antagonist is selected from the group consisting of MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof.

11. The method of claim 10, wherein the therapeutically effective amount of the GHRH antagonist is administered parenterally or enterally.

12. The method of claim 10 or 11, wherein the subject is obese or at risk of obesity.

13. The method of any one of claims 10-12, wherein the subject has a body mass index (BMI) of over 25.

14. The method of any one of claims 10-12, wherein the subject has a body mass index (BMI) of between 25 and 30.

15. The method of claim 10-12, wherein the subject has a body mass index (BMI) of over 30.

16. The method of claim 14, wherein the subject is human.

17. A method for reducing or inhibiting reduce triglyceride-rich-lipoproteins (TRL) levels in a subject in need thereof comprising administering to the subject an effective amount of a GHRH antagonist or a pharmaceutical composition containing the GHRH antagonist to reduce TRL levels in the subject.

18. The method of claim 17, wherein the subject has pre-diabetes, type I diabetes, type II diabetes, gestation diabetes, is obesity, or a combination thereof.

19. The method of claim 17 or 18, wherein the GHRH antagonist is selected from the group consisting of MIA-602, AVR-235, AVR-333, AVR-352, AVR-353, AVR-354, AVR-104, AVR-107, AVR-116, AVR-120, AVR-201, AVR-234, AVR-321, AVR-322, AVR-542, AVR-543, AVR-552, AVR-553, AVR-620, and combinations thereof.
